(19) United States
(12) Patent Application Publication
(10) Pub. No.: US 2009/0193559 A1
(43) Pub. Date: Aug. 6, 2009

(54) Method and apapratus to cover eyes while sleeping

(76) Inventor: Sharon A. Ritchey, Cave Creek, AZ (US)

Correspondence Address: TOD R NISSLE, PO BOX 55630, PHOENIX, AZ, 85078, US

(21) Appl. No.: 12/012,374

(22) Filed: Feb. 1, 2008

(51) Int. Cl. A61F 9/00

(52) U.S. Cl. 2/15

(57) ABSTRACT

A method to cover the eyes of an individual while sleeping. The method includes the step of providing a floating mask unit comprising a strap sized to extend over an upper front of the individual's head, down the sides of the head over at least a portion of the ears, temporal bones and sphenoid bones, and to a nape of the individual's neck, and comprising a mask foldably, pliably attached to the strap to move between at least two operative positions, a first operative deployed position hanging loosely and freely and extending downwardly from the strap over the forehead and covering the eyes of the individual, and a second operative stored position folded upwardly and extending upwardly and rearwardly from the strap on top of the head of the individual. The method also includes the steps of mounting the mask unit on the head by mounting the strap to extend over the upper front of the individual's head, down the sides of the individual's head over at least a portion of the ears, temporal bones and sphenoid bones, and down the sides of the head to the nape of the neck, and moving the mask to the first operative position extending downwardly over the eyes of the individual; sleeping; waking; and, moving the mask from the first to the second operative position.

This invention pertains to a method and apparatus for covering and shielding the eyes.

More particularly, the invention pertains to a method and apparatus that covers the eyes with a mask that facilitates sleeping with and utilizing the mask.

A variety of masks are known and available for covering the eyes of an individual. It would be desirable to provide such a mask that is not suggested by a trend in the use or construction of masks, that if anything runs contrary to current trends in mask production and use, that is unpredictable, that is not readily produced by simple substitution of known mask components or processes, and that facilitates being able to wear the mask while resting.

Therefore, it an object of the present invention to provide an improved method and apparatus for covering the eyes of an individual.

A further object of the invention is to provide a mask assembly that is conveniently utilized during sleep.

These and other, further and more specific objects and advantages of the invention will be apparent to those of skill in the art from the following detailed description thereof, taken in conjunction with the drawings, in which:

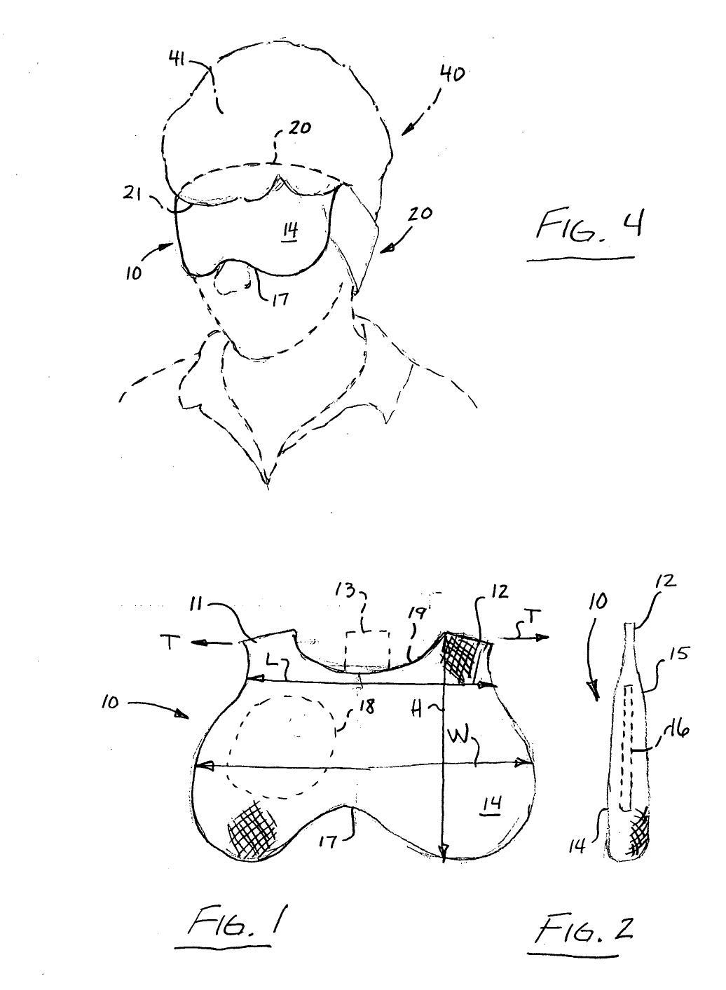

FIG. 1 is a front view illustrating the mask portion of a mask assembly constructed in accordance with the principles of the invention;

FIG. 2 is a side view illustrating the mask portion of FIG. 1;

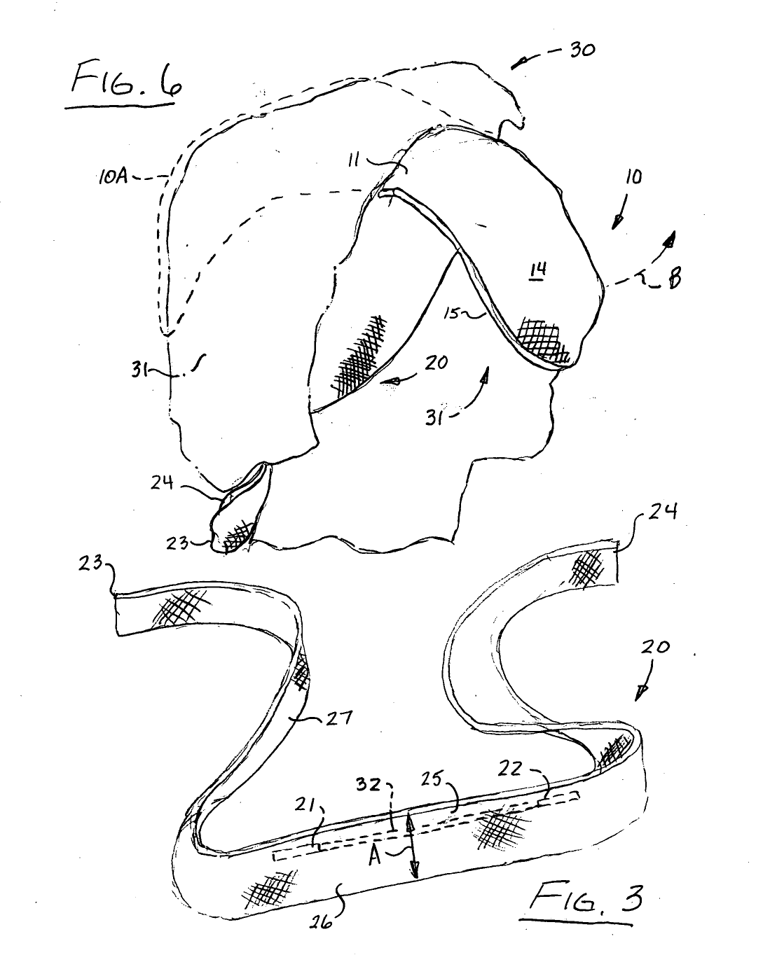

FIG. 3 is a perspective view illustrating the strap portion of a mask assembly constructed in accordance with the principles of the invention;

FIG. 4 is a perspective view illustrating the mask assembly of the invention worn by an individual to cover the individual's eyes;

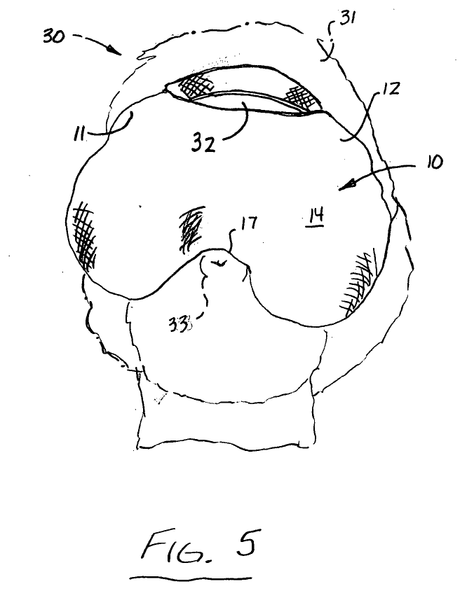

FIG. 5 is a front view illustrating the mask assembly of the invention worn by another individual and depicting the spaced apart attachment points of the mask portion to the strap portion to eliminate the transmission of tensile, or stretching, forces from the strap portion of the mask portion; and,

FIG. 6 is a side view further illustrating the mask assembly and individual of FIG. 5.

Briefly, in accordance with the invention, I provide an improved method to cover the eyes of an individual while sleeping. The method comprises the step of providing a floating mask unit comprising a strap sized to extend over the upper front of the individual's head, down the sides of the head, and to a nape of a neck of an individual; and, a mask foldably, pliably attached to the strap to move between at least two operative positions, a first operative deployed position hanging loosely and freely and extending downwardly from the strap over the forehead and covering the eyes of the individual, and a second operative stored position folded upwardly and extending upwardly and rearwardly from the strap on top of the head of the individual. The method also includes the step of mounting the mask unit on the individual's head by mounting the strap to extend over the upper front and sides of the head to the nape of the neck; and, moving the mask to the first operative position extending downwardly over the eyes of the individual. The method also includes the steps of sleeping; waking; and, moving the mask from the first to the second operative position.

Turning now to the drawings, which depict the presently preferred embodiments of the invention for the purpose of illustrating the practice thereof, and not by way of limitation of the scope of the invention, and in which like characters refer to corresponding elements throughout the several views, FIG. 1 illustrates the mask portion 10 of the floating mask unit of the invention. FIG. 3 illustrates the strap portion 20 of the mask unit of the invention.

The mask portion 10 includes front face 14 and rear face 15. When the mask unit is worn on the head of an individual, rear face 15 is adjacent and contacts the face of the individual. Mask portion 10 has a width, indicated by arrows W, and a height, indicated by arrows H, sufficient to cover the eyes of an individual when the mask unit is worn on the head of an individual. The mask portion 10 can be constructed of any desired material or materials, but preferably consists of a hollow shell constructed of a soft, lightweight fabric. The shell is filled with goose down or another lightweight fabric or material. This construction makes the mask soft, cushiony, air permeable, and lightweight so that the mask is not uncomfortable in the event an individual wearing the mask and sleeping rolls—face down—onto the mask. The goose down or other lightweight fill permits at least some air to flow through the fill or intermediate the mask portion 10 and user's face to facilitate the user's breathing while laying on the mask. Further, if desired, the mask portion 10 can be provided with one or more openings and pockets 16 into which cold or hot packs 18 can be inserted.

Mask portion 10 also includes one or more pliable, bendable flaps or arms 11 to 13 that are sewn or otherwise attached to the strap portion 20 at selected locations 21 and 22 (FIG. 3). If desired, arms 11 to 13 can be removably secured to strap portion 20 with VELCRO® fastener material or other fastening means. Top edge 19 of mask portion 10 can also be configured to be attached to strap portion 20 along the area indicated by dashed lines 25 in FIG. 3. This would enable, if desired, substantially the entire top edge of mask portion 10 to be connected to strap portion 20.

One important feature of the invention comprises attaching mask portion 10 to strap portion 20 such that when the strap portion 20 is mounted on the head of an individual, the strap portion 20 does not laterally tension mask portion 20 across the forehead and face of the individual. Consequently, in the embodiment of the invention illustrated in FIGS. 1 to 6, this is achieved by attaching only parts of the top of mask portion 10 to strap portion 20 and by not attaching mask portion 10 to strap portion 20 along substantially the entire length, indicated by arrows L, of the mask portion 10. Instead, mask portion 10 is attached to strap portion 20 at least two separate, spaced apart locations by spaced apart arms 11 and 12.

Further, when the strap portion is mounted on the head of an individual, strap portion 20 does not apply lateral tensile forces, indicated by arrows T, to mask portion 10 that tend to pull arms 11 and 12 apart and, accordingly, tend to press the top edge 19 of the mask portion 10 against the forehead of the individual. Instead, there are no tensile forces T generated by strap portion 20 and acting on arms 11 and 12 and the top portion of mask portion 10 and, accordingly, top edge 19 and the remainder of mask portion 10 hangs freely and loosely from strap portion 20. When an individual's head is upright, in the manner illustrated in FIGS. 4, 5 and 6, mask portion 10 hangs freely downwardly over the eyes and face of an individual. Since mask portion 10 hangs freely downwardly over the eyes and face of an individual, since mask portion 10 is preferably lightweight and soft, and since strap 20 does not laterally tension mask portion 10, mask portion 10 does not generate any pressure points of significance on the sinus areas, forehead, or other areas of an individual's head, making the floating mask unit of the invention comfortable to wear for extended periods of time, particularly while sleeping.

Similarly, when, in another embodiment of the invention, upper edge 19 or substantially the entire length of the top edge portion of mask portion 10 are attached to strap portion 20, then at least the upper portion of mask portion 10, and if necessary strap portion 20, is constructed such that mask portion 10 can, without pressing strap portion 20 against the head of the individual, freely pivot upwardly from the position illustrated in FIG. 5 to a position indicated in FIG. 6 on top of an individual's head. In one construction of the invention, the top and upper edge 19 of the mask portion 10 are constructed from a knit material that can readily stretch so that moving mask portion 10 from a position over a user's face to a position on top of the user's head does not press the strap portion 20 against the user's head along any portion of the length of edge 19 when substantially the entire length of the top edge portion of mask portion 10 is attached to strap portion 20. When mask portion 10 is pivoted upwardly from a position over a user's face to a position on top of the user's head, the position of strap portion 20 is not altered, i.e., a section of strap portion 20 that extends over the upper front of the skull is not pulled or tilted outwardly away from the user's head. Strap portion 20 remains in contact with the upper front of the skull. This is also the case with the embodiment of the invention illustrated in FIGS. 1 and 2.

Mask portion 10 can, as noted, be detachably secured to strap portion 20 (with hook and loop fastener material or other fastener material); and, can be secured to strap portion 20 such that mask portion 10 can be attached to strap portion 20 at different selected points.

The strap portion 20 of the invention illustrated in FIG. 3 includes ends 23 and 24, intermediate portion 25, front 26, and back 27. Arm 11 is sewn or otherwise connected to the front 26 of strap portion 20 at the location noted by dashed lines 21. Arm 12 is sewn or otherwise connected to the front 26 of strap portion 20 at the location noted by dashed lines 22.

Strap portion 20 can be fabricated from an elastic material as long as portion 20 does not, when mounted on the head of an individual, create tensile forces that act to pull arms 11 and 12 apart and press the top edge 19 against the forehead of an individual. However, strap portion 20 preferably is not made of an elastic material and, rather, is fabricated from a soft substantially non-elastic material like cotton.

While the width A of strap portion 20 can vary as desired, the width A is normally at least one-half inch, preferably at least three-quarters of an inch, and most preferably at least one inch. The width A is also normally less than about four inches, preferably less than three inches, and most preferably less than two inches. Dimensions given herein are to within one-eighth of an inch. For example, a dimension of two inches means two inches plus or minus one-eighth of an inch. Strap portion 20 is not comprised of a piece of string or some other material have a width less than one-quarter of an inch because such tends to dig into the skin and is uncomfortable.

Strap portion 20 can be a continuous piece of material fitted to the head of an individual. The presently preferred embodiment, however, comprises a “tie” strap portion 20 that is secured on the head of an individual by tying together free ends 23, 24 in the manner illustrated in FIG. 6. Or, strap portion 20 includes free ends 23, 24 that are adjustably connected to each other using VELCRO fastener material attached to each free end 23, 24; or, by using other fastener components connected to a free end(s) 23, 24. The use of free ends 23, 24 that can be tied or otherwise adjustably affixed to one another enables the strap portion 20 to be comfortably, and not too tightly, mounted on the head of an individual.

When strap portion 20 is mounted on the head of an individual, the location of strap portion 20 is critical. Strap portion 20 must, as illustrated in FIGS. 4 to 6, extend from the nape of the neck; up and at least partially over the ears, temporal bones, and sphenoid bones of an individual, and across the front and top of the individual's skull. This means that strap portion 20 typically extends over the location of the hairline that extends over and across the front of the skull above the forehead; or, extends over the top front of the skull behind the hairline and between the hairline and the coronal suture of the individual (or over the top front of the skull on top of or behind location of the hairline that existed before the individual lost his hair). Importantly, the strap portion 20 does not extend in a plane that is generally parallel to the ground and, accordingly, does not extend across the forehead, the sides of the skull, and around the occipital bone at the back of the skull. In FIG. 6, strap 20 completely covers the ears of the individual.

One reason this positioning of the strap portion 20 is important is that the knot or other material overlap created by tying (or otherwise attaching) free ends 23 and 24 is nested in the hollow located at the nape of the neck just below the back of the skull. When the knot is in this position, it is less likely to create a pressure point when an individual is sleeping on his back.

Another reason the positioning of the strap portion 20 is important is that the intermediate portion 25 of strap portion 20, in the manner illustrated in FIGS. 4 and 6, extends at least partially over and presses down on a portion of the frontal bone that forms part of the top of the skull and that extends from the forehead boss back toward the coronal suture that extends across the top of the skull. Such pressure tends to be more comfortable than pressure directed against the forehead in a direction that is generally normal to the forehead.

FIG. 4 illustrates the floating mask unit of the invention mounted on the head 40 of a woman. Strap portion 20 extends from the nape of the neck, over the woman's ears, and across the hairline of the woman. The bangs 21 of the woman's hair 41 hang down over the top of strap portion 20. The lower arcuate, concave edge 17 of mask portion 10 extends up and over the nose 33 of the woman.

In use of the floating mask unit of the invention, the mask unit is mounted on the head 30 of an individual in the manner illustrated in FIGS. 5 and 6. Free ends 23 and 24 are tied together at the nape of the neck. Strap portion extends from the nape of the neck up over the ears and hairline of the individual. As used herein, the “traversing” portion of an individual's head is the front part of an individual's head that extends from one side of the head to the other side of the head and that is positioned between the generally vertically oriented forehead of the individual (when the individual is standing erect) and the generally horizontally oriented top of the individual's head (when the individual is standing erect). The traversing portion extends over the frontal bone between the forehead boss and the coronal suture. The traversing portion is, however, spaced apart from the coronal suture. For most individuals, the hairline is located in this traversing portion of the skull. In some individuals, the hairline may be located more on the vertically oriented forehead of the individual. If the intermediate portion 25 of strap portion 20 extends only across the vertically oriented forehead of an individual, intermediate portion 25 will tend to slide down the face of the individual, which is undesirable. As a result, in order for intermediate portion 25 to have some “purchase”, it must at least in part extend over the traversing portion of an individual's skull in the manner illustrated in FIG. 6 because the traversing portion is not substantially vertically oriented, but is, at a minimum, sloped and has a horizontally oriented component. Further, the width of strap portion 20 means that strap portion 20 often tends to extend a distance to either side of the hairline, and that, consequently, the section of portion 20 that extends from the hairline toward the top of the individual's head is contacting an area of the top of the skull that is (when the individual is standing) more horizontally oriented than the forehead of the individual.

In FIGS. 5 and 6, mask portion 10 is in the deployed position and hangs freely over and floats on the face 31 of the head 30 of the individual and covers the individual's eyes (not visible). After the mask unit is secured on the individual's head 30, the individual sleeps. After the individual wakes, the individual can, if desired, manually “flip” or move the mask portion 10 in the direction of arrow B from the freely hanging deployed position illustrated in FIG. 6 to a storage position on top of the individual's head and hair 31, which storage position is indicated in FIG. 6 by dashed lines 10A. Arms 11 and 12 preferably comprise pliable fabric which permits arms 11 and 12 to bend readily and function like hinges to facilitate movement of mask portion 10 between the deployed and stored positions. Arms 11 and 12 can be constructed in any desired manner from any desired material to fulfill this hinge function. Soft fabric hinges or hinges made from similar materials are preferred because they are lightweight, they do not create pressure points of any significance on the head of an individual, and they feel comfortable.

Having described my invention in such terms as to enable those of skill in the art to make and user the invention, and having described the presently to make and use of the invention, and having described the presently preferred embodiments thereof, I Claim:

1. A method to cover the eyes of an individual while sleeping, comprising the steps of
(a) providing a floating mask unit comprising
(i) a strap sized to extend over an upper front of the individual's head, down the sides of the head over at least a portion of the ears, temporal bones and sphenoid bones, and to a nape of the individual's neck,
(ii) a mask foldably, pliably attached to said strap to move between at least two operative positions,
a first operative deployed position hanging loosely and freely and extending downwardly from said strap over the forehead and covering the eyes of the individual, and a second operative stored position folded upwardly and extending upwardly and rearwardly from said strap on top of the head of the individual;
(b) mounting said mask unit on the head by
(i) mounting said strap to extend over the upper front of the individual's head, down the sides of the individual's head over at least a portion of the ears, temporal bones and sphenoid bones, and down the sides of the head to the nape of the neck, and
(ii) moving said mask to said first operative position extending downwardly over the eyes of the individual;
(c) sleeping;
(d) waking; and,
(e) moving said mask from said first to said second operative position.
